(19) United States
(12) Patent Application Publication
(10) Pub. No.: US 2021/0269867 A1
(43) Pub. Date: Sep. 2, 2021

(54) DEVICE FOR DETECTING NUCLEIC ACID AMPLIFICATION REACTION PRODUCTS IN REAL TIME

(71) Applicant: LOGOS BIOSYSTEMS, INC., Anyang-Si, Gyeonggi-Do (KR)

(72) Inventor: Neoncheol JUNG, Anyang-Si, Gyeonggi-Do (KR)

(21) Appl. No.: 17/048,753

(22) PCT Filed: Apr. 17, 2019
(86) PCT No.: PCT/KR2019/004602

§ 371 Date: Oct. 19, 2020

(30) Foreign Application Priority Data
Apr. 17, 2018 (KR) 10-2018-0044349

(51) Int. Cl. C12Q 1/6844; G01N 21/03; G01N 21/64

(52) U.S. Cl. CPC C12Q 1/6844; G01N 21/0332; G01N 2201/062; G01N 21/645; G01N 21/6428

(57) ABSTRACT

The present invention relates to an apparatus for measuring a reaction product generated by a nucleic acid amplification reaction from two or more samples processed simultaneously or independently. In particular, the present invention relates to a nucleic acid amplification reaction product real-time detection apparatus comprising a nucleic acid amplification reaction unit, an optical detection unit, and a control unit, characterized in that the mirror and the focusing lens are relatively rotated to inject excitation light into a specific reaction chamber. Accordingly, the apparatus of the present invention can measure a nucleic acid amplification reaction product of a sample in real-time, regardless of whether any other samples are put in or out.

### TECHNICAL FIELD

The present invention relates to an apparatus and a method for real-time detection of a nucleic acid amplification reaction product.

### BACKGROUND ART

Nucleic acid amplification is an important technology for research, medical, and industrial purposes, and polymerization chain reaction (PCR) or isothermal amplification has been used for the amplification. The amplified nucleic acid was usually detected by electrophoresis, but a method of real-time detecting a nucleic acid amplified using a fluorescent label has been widely introduced. The real-time amplification method has the advantage of being able to detect multi-targets in one tube using different fluorescent labels and to estimate the concentration of nucleic acids before the said reaction. An intercalating dye capable of displaying fluorescence can be used as a fluorescent labeling. Further, a fluorescent label may use the property of fluorescence expression when a probe consisting of a fluorescence-quencher is cut off by polymerase during amplification. The amplification reaction of nucleic acids is performed in a closed reaction chamber to prevent cross-contamination between samples and evaporation due to amplification temperature. As a typical closed reaction chamber, a plastic tube or a tube array has been used. Various fluorescence detection devices have been developed to precisely measure the fluorescence generated in a sample contained in the tube. Since the amplification reaction of nucleic acids is usually carried out under a temperature-cycling or high temperature isothermal conditions, the tube is placed in a conductive metal block for convenient heat transfer and the top of the tube is usually heated higher than the reaction temperature to prevent evaporation of the sample. In addition, it is highly required to individually detect fluorescent signals generated from a plurality of tubes.

U.S. Pat. No. 5,928,907 teaches an optical device for real-time detecting fluorescence of amplified nucleic acids using image files converted by an optical fiber cable, a lens coaxially arranged to the said fiber cable, and a CCD (Charge Coupled Device) array that converts the intensity of light into an electrical signal. The commercially available Perkin Elmer 7700 (Applied BioSystems) was used to monitor fluorescence in different tubes by sequentially placing fiber optic cables on top of different reaction chambers to detect fluorescence in a plurality of closed reaction chambers. Wittwer et al. (1997) introduced a commercial optical device, LightCycler (trademark), which places a microtube (tube) containing a sample in a circular carousel and arranges the said tube in the detection optical system in consecutive order while circularly rotating the sample. The multiple samples are placed on a carousel rotating in a chamber in which heated air is circulated, instead of a metal block, and fluorescence is detected when individual samples are placed on top of an optical system composed of an illumination system and a detection system during rotation. U.S. Pat. No. 5,675,155 discloses a structure using a scanning mirror and a scanning lens to detect electromagnetic signals radiated from a plurality of samples contained in a capillary electrophoresis device. However, this method has a disadvantage in that it can be implemented only by using expensive parts such as a galvano scanner and a scanning lens. In addition, U.S. Pat. No. 6,818,437 discloses a construct of field lens for real-time detection of nucleic acid amplification reaction signals for a plurality of samples, wherein the field lens is arranged on top of each sample and focuses a beam on individual samples. A Fresnel lens can be used as a field lens, and the signal of an individual sample can be separated and detected since the light passing through the Fresnel lens is not mixed with a fluorescent signal and is individually imaged by the array detector. Further, KR Patent Pub. No. 10-2009-0000474 discloses a device to efficiently detect fluorescence from a nucleic acid amplification reaction using a polarizer to efficiently separate incident light and excitation light, a polarizing beam splitter, and a polarization converting system, and teaches a Fresnel lens to separate and then detect fluorescent signals of a plurality of individual samples as shown in U.S. Pat. No. 6,818,437.

In clinical sites, it is often necessary to analyze samples in the order in which patient samples are received, rather than to analyze all patient samples at once. When samples from patients visiting a hospital are collected and analyzed at once, there would be a problem in that some patients may receive the diagnosis results late. On the other hand, to analyze each sample in the order of patients' visiting a hospital results in an increase in analysis time and costs due to the poor operation efficiency, since it is not possible to analyze the sample during the use of the device for a previous sample. Therefore, an ideal real-time gene amplification for diagnostic purposes is to analyze a large number of samples at once while the samples are processed individually. The prior arts mentioned above, however, disclose amplification and detection of nucleic acids for a number of samples at once, and it is difficult to independently analyze individual samples having different initiation time points of the nucleic acid amplification reaction. For example, although a number of samples can be amplified and analyzed using a metal block containing a tube array in the form of a well plate, a new sample cannot be inserted in the middle for analysis since all samples are amplified and detected at once. In case a sample is accommodated in a rotating carousel, individual temperature control for multiple samples is not allowed. Further, if a sample is inserted during the process, fluorescence detection of the other samples under process should be stopped for a while, resulting in inaccurate results. In this regard, we, inventors have tried to develop a device and a detection method capable of real-time detection of nucleic acid amplification not only for a plurality of samples but also for individual samples, and have completed the present invention. Therefore, the present invention can be used to monitor the nucleic acid amplification reaction in real time irrespective of the loading and unloading of a sample during the reaction.

### SUMMARY OF INVENTION

### Technical Problem

The present invention provides a device for real-time detection of nucleic acid amplification, which simultaneously and/or independently performs a nucleic acid amplification reaction for a plurality of samples and measures it in real time.

Another object of the present invention is to provide a method for real-time detection of nucleic acid amplification, which simultaneously and/or independently performs a nucleic acid amplification reaction for a plurality of samples and measures it in real time.

### Solution To Problem

In order to achieve the above object, the present invention provides an apparatus for measuring a nucleic acid amplification reaction product in real time, characterized by comprising,

(i) a nucleic acid amplification reaction unit comprising two or more reaction chambers equipped with a sample receiving unit, and a temperature control unit for heating and cooling the reaction chamber;

(ii) an optical detection unit for measuring a nucleic acid amplification reaction product, comprising a light source for irradiating light to a sample in the reaction chamber, a focusing lens for providing excitation light irradiated from the light source to a specific reaction chamber, a mirror for switching the light path, and a photodetector and a light receiving lens for detecting fluorescence emitted from the sample, wherein characterized in that the mirror and the focusing lens are relatively rotated to transmit the excitation light into the specific reaction chamber; and

(iii) a control unit connected to the optical detection unit and the nucleic acid amplification reaction unit through bidirectional communication, comprising an input and an output units and a central processing unit for analyzing and storing the intensity of the fluorescence signal of the nucleic acid amplification reaction product transmitted from the optical detection unit.

According to an embodiment of the present invention, an apparatus for measuring a nucleic acid amplification reaction product in real time is provided, wherein the temperature control unit is composed of a thermoelectric element heated and cooled and a metal block, and the reaction chamber is accommodated in the metal block.

According to another embodiment of the present invention, an apparatus for measuring a nucleic acid amplification reaction product in real time is provided, wherein the temperature control unit is composed of a gas supply device for heating and cooling a reaction chamber.

According to yet another embodiment of the present invention, an apparatus for measuring a nucleic acid amplification reaction product in real time is provided, wherein the light source detection unit further comprises a filter and a beam splitter between the light source and the mirror or between the mirror and the light-receiving lens.

According to yet another embodiment of the present invention, an apparatus for measuring a nucleic acid amplification reaction product in real time is provided, wherein the light source is a light-emitting diode array or a light-emitting diode matrix which comprises blue, green or red LEDs.

According to another embodiment of the present invention, an apparatus for measuring a nucleic acid amplification reaction product in real time is provided, wherein the nucleic acid amplification reaction is a PCR reaction or an isothermal amplification reaction.

According to another embodiment of the present invention, an apparatus for measuring a nucleic acid amplification reaction product in real time is provided, wherein the nucleic acid amplification reaction of the samples in each reaction chamber is initiated at a different time point, so that the nucleic acid amplification reaction step of each sample is different from each other.

### Advantageous Effects of Invention

The apparatus for real-time detection of nucleic acid amplification reaction products according to the present invention can detect in real time not only the nucleic acid amplification reaction products performed simultaneously in the reaction chamber, but also the nucleic acid amplification reaction products in samples having different starting points and process steps of the nucleic acid amplification reaction. Therefore, it is possible to immediately process samples in need of analyzing their nucleic acid amplification reaction products without having to wait to process multiple samples at the same time in clinical sites.

### BRIEF DESCRIPTION OF THE DRAWINGS

FIG. 1 is a block diagram showing the main components of the apparatus of the present invention.

FIG. 2 schematically shows an apparatus according to an embodiment of the present invention.

FIG. 3 shows the structure of a device (Bio-rad) according to the prior art.

### DESCRIPTION OF EMBODIMENTS

Hereinafter, preferred embodiments of the present invention will be described in detail with reference to the accompanying drawings. The following examples are for explaining the present invention, and the scope of the present invention is not limited thereto, and modifications that can be easily substituted and changed by those skilled in the art will be included in the scope of the present invention.

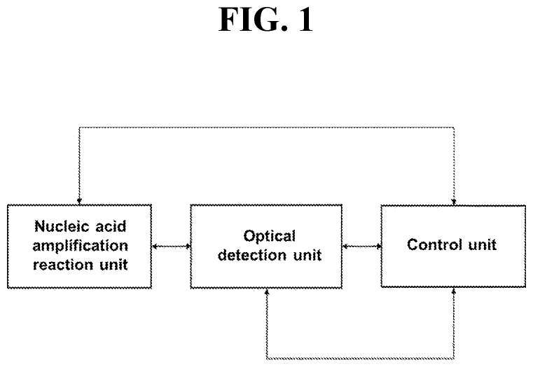

FIG. 1 is a block diagram showing the main components of the apparatus of the present invention. As shown in FIG. 1, the real-time detection device for a nucleic acid amplification reaction product of the present invention comprises a nucleic acid amplification reaction unit, an optical detection unit, and a control unit, wherein the control unit controls the nucleic acid amplification reaction unit and the optical detection unit through a two-way communication network, and computes and stores fluorescence detection result of the nucleic acid amplification reaction product. Therefore, the control unit may further include a central processing unit (CPU) and an input/output device.

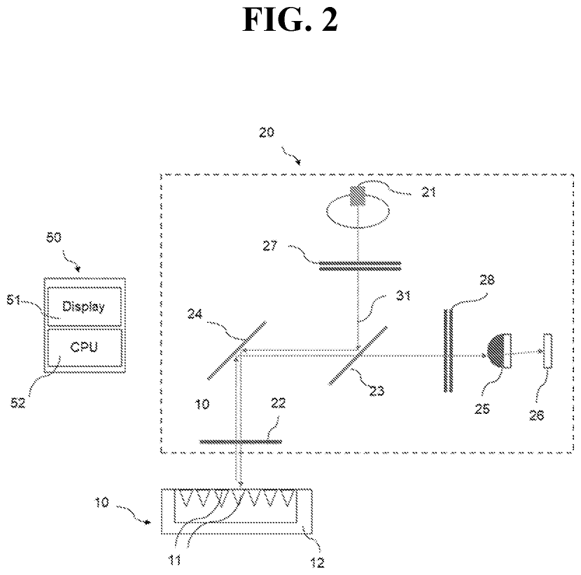

FIG. 2 schematically shows an apparatus for measuring fluorescence of a nucleic acid amplification reaction product in real time according to the present invention.

As shown in FIG. 2, the device of the present invention comprises,

(i) a nucleic acid amplification reaction unit (10) comprising two or more reaction chambers (11) equipped with a sample receiving unit (11) for accommodating biological samples in need of a nucleic acid amplification reaction, and a temperature control unit (12) for heating and cooling the reaction chamber;

(ii) an optical detection unit (20) for measuring the nucleic acid amplification reaction, comprising the light source (21), one or more filters (27, 28), mirrors (23, 24), a focusing lens (22) for transmitting excitation light irradiated from the light source (31) into a targeting specific reaction chamber (11), a light-receiving lens and a photodetector (26),

characterized by being equipped with a driving device for relatively rotating the mirrors (23, 24) and the focusing lens (22); and

(iii) a control unit 50.

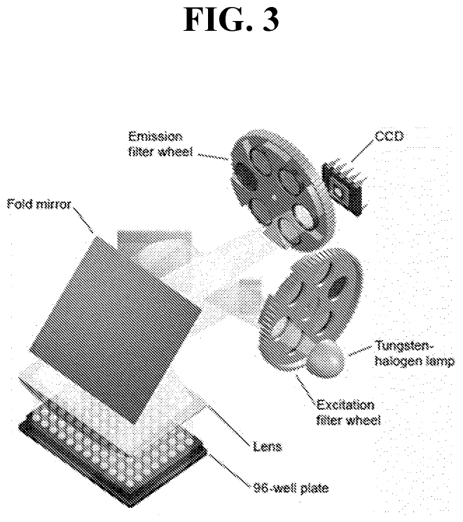

FIG. 3 shows the structure of a commercially available nucleic acid amplification reaction detection device (Bio-rad), wherein a filter applied to excitation light and emitted fluorescence is provided in the form of a wheel.

In one embodiment of the present invention, the light source irradiates an excitation light (31) for emitting fluorescence from a nucleic acid amplification product of a sample in the reaction chamber (11), wherein the light source is a blue LED, a red LED, or a green LED (light-emitting diode) or a halogen lamp. The light emitting diode according to an embodiment of the present invention may be a light emitting diode array or a light emitting diode matrix. Further, in an embodiment of the present invention, the photodetector is a photodiode, a PMT, a CCD camera, or a CMOS camera, and the light source, filter, mirror, and focusing lens may each be provided with a driving device. In an embodiment related to this, each of the light source, filter, mirror, and focusing lens may be accommodated in a housing additionally provided together with their driving devices. In one embodiment of the present invention, the driving device or actuator may be a solenoid, a relay, a DC motor, and a stepper motor, but is not limited thereto. In the present invention, the driving device and the actuator are understood to have the same meaning and are used interchangeably. According to an embodiment of the present invention, when a driving device or an actuator is provided, the light source, filter, mirror, and focusing lens may be disposed at a specific position according to a command signal transmitted from a control unit through a two-way communication network.

According to FIG. 2, the optical path of the excitation light irradiated from the light source (21) and passed through the filter (27) is switched in any direction by the first mirror (23) disposed on the beam transmitting path and then irradiated to the second mirror (24). The filter is preferably a band pass filter and may be disposed at any position between the light source and the first mirror (23) or between the first mirror (23) and the light receiving lens (25). The band filter according to an embodiment of the present invention refers to a filter that selectively passes only light of a specific wavelength. According to an embodiment of the present invention, a dichroic beam splitter (not shown in the drawings) may be further provided to separate excitation light and fluorescence.

In addition, according to an embodiment of the present invention, the said band filter (28) is a filter having a property of transmitting only specific fluorescence from the fluorescence emitted from the sample and the reflected light, whereby any reflected light other than the fluorescence emitted from the sample is blocked. The light-receiving lens (25) according to the embodiment of the present invention transmits the fluorescence transmitted through the band filter (28) to the photodetector (26), and the fluorescence detection result converted into an electrical signal by the photodetector (26) is transmitted to the control unit (50) by a two-way wired/wireless communication network. According to an embodiment of the present invention, the two-way wireless communication method includes bluetooth, 3G/4G/LTE, wifi, ZigBee, NFC (Near Field Communication), RFID (Radio-Frequency Identification), and LoRa (Long Range), but is not limited thereto. The optical signal information as a result of fluorescence detection of the nucleic acid amplification reaction product transmitted to the control unit (50) is processed and stored in the central processing unit (51), and each quantity of the nucleic acid amplification reaction product of the sample in each reaction chamber can be analyzed independently. In the present invention, the band filters (27, 28) may be appropriately selected in accordance with the types and characteristics of the sample, the light source, and the fluorescent dye, and be accommodated in a filter housing so as to be detachable.

According to an embodiment of the present invention, the band filter may be manufactured such that at least one band filter is provided in a rotatable substrate (revolving stage) so that any band filter can be selected. In addition, an actuator which drives according to a command signal from a control unit may be further provided on the rotatable substrate with the band filter.

In one embodiment of the present invention, the temperature control unit (12) in the nucleic acid amplification reaction unit (10) is composed of a heating and cooling element, for example, a thermoelectric element and a conductor such as a metal block, or composed of a gas supplying device that is heated and cooled. When the conductor is provided, the conductor has a plurality of support grooves or hall structures to support the reaction chamber for accommodating the sample therein. In addition, the conductor may be equipped with a sensor, and the temperature of the thermoelectric element may be controlled by a temperature detected by the sensor. In addition, the temperature control unit (12) may include a processing unit, a power control unit, and a power source in addition to the heating and cooling elements and sensors. The processing unit can initiate heating and cooling by instructing the power control unit to supply power from the power source to the heating and cooling elements. In addition, the nucleic acid amplification reaction unit (10) may be housed by a heat insulating member, for example, ceramic, which blocks the heat of the nucleic acid amplification reaction unit from being transferred to the outside. In addition, a sensor capable of detecting deformation of the sample due to heat generation of the nucleic acid amplification reaction unit (10) may be further provided.

In another embodiment of the present invention, the reaction chamber (11) has a closed bottom and an open top. The reaction chamber (11) is, for example, a tube, a well plate having a plurality of wells, and a petri dish, a slide, a filter, a Terasaki plate, or a PCR plate. In addition, the upper part of the reaction chamber may be detachably attached to a cap member or sealed with a sealing material such as tape, thereby providing a closed system to the reaction chamber during a nucleic acid amplification reaction. The cap member and the sealing material should be light-transmitting and capable of transmitting excitation light and fluorescence emitted from the sample. Therefore, in the present invention, the cap member and the sealing material may be light-transmitting silicone, urethane, transparent PVC, or a mixture thereof, but are not limited thereto. The reaction chamber may further include a heat sink, and an isolation groove may be provided between each well constituting the reaction chamber to prevent contamination by a solution. In addition, the thermoelectric device may be a peltier device.

In another embodiment of the present invention relating to this, the temperature control unit (12) is configured as a gas supply device. In this case, each of the reaction chambers or two or more reaction chambers may be separated by a partition wall blocking gas from communicating with each other, and may be heated and cooled by individually supplied gas.

In an embodiment according to the present invention, the lens (22) and the mirrors (23, 24) in the optical detection unit (20) is arranged so as to be relatively rotatable to irradiate excitation light into the specific reaction chamber (11) and to receive fluorescence emitted from the sample. That is, the mirrors (23, 24) and the lens (22) are provided to be rotated relatively 360° without limitation in the vertical and horizontal directions so as to be focused on a specific reaction chamber, and thus nucleic acid amplification reaction products can be measured independently for each of the reaction chambers. In addition, when the driving device or actuator according to an embodiment of the present invention is configured in the mirror and the lens, it can be moved and rotated according to a command signal from the control unit. Accordingly, it is possible to independently monitor each reaction chamber even when a sample is provided to the reaction chamber at a time difference to perform the nucleic acid amplification reaction at different starting points of the nucleic acid amplification reaction.

Referring to FIG. 2, in an embodiment of the present invention, the control unit (50) of the present invention may comprise an input/output unit (52) and a central processing unit (51) for calculating and storing detection results from the optical detection unit (20). The central processing unit of the control unit (50) may be informed of temperature information and location information of each reaction chamber in which the nucleic acid amplification reaction is performed, information on the start time and step of the nucleic acid amplification reaction, information on the wavelength of excitation light and fluorescence, and information on the relative arrangement of the mirrors (23, 24) for focusing on a specific reaction chamber from a sensor capable of bidirectional communication (not shown in the drawing) provided in each nucleic acid amplification reaction unit (10) and the optical detection unit (20). In addition, the control unit (50) may detect a nucleic acid amplification reaction process in a specific target reaction chamber (10) at a specific time point or on a regular basis, for example, after the end of one cycle, according to an input value or a preset value.

Hereinafter, a process for real-time detection of the nucleic acid amplification reaction product of the present invention will be described.

First, the beam irradiated from the light source (21) reaches the first mirror (23) through the filter (27). The filter (27), preferably a bandpass filter, transmits the excitation light from the beam irradiated from the light source (21). The excitation light is irradiated to the second mirror (24) by the first mirror (23) and is irradiated again to a targeting specific reaction chamber through the focusing lens (22).

Fluorescence emitted from the sample subjected to the nucleic acid amplification reaction in the reaction chamber reaches the light-receiving lens (25) and the photodetector (26) via the second mirror through the focusing lens (22). Since only the fluorescence of a predetermined wavelength range is transmitted through the first mirror (23) and the band filter (28), the camera (26) selectively detects only the specific fluorescence. In one embodiment of the present invention, the fluorescence detection result of each nucleic acid amplification reaction product detected by a photodetector, for example, a photodiode, is transmitted to and analyzed by the control unit (50) interfacing by wired or wireless two-way communication.

In an embodiment of the present invention, the control unit (50) may store a result of the detected nucleic acid in a computer-readable medium or a cloud system, and compare the result with records stored in other databases. In addition, in an embodiment of the present invention, the computer may be personal digital assistants (PDAs), smart phones, tablets or any other portable or mobile electronic device, and be any other forms such as computers embedded in devices with suitable processing capabilities. Such computers may include one or more input and output units.

### INDUSTRIAL APPLICABILITY

According to the apparatus for real-time detection of a nuclear amplification reaction product of the present invention, it is possible to simultaneously and independently perform and detect a nucleic acid amplification reaction for a plurality of different biological samples in clinical practice. Accordingly, the apparatus of the present invention can be usefully used in clinical sites requiring immediate execution and monitoring of nucleic acid amplification reactions.

1. A device for real-time detection of nucleic acid amplification reaction products, comprising:
a nucleic acid amplification reaction unit (10) comprising two or more reaction chambers (11) equipped with sample receiving units, and a temperature control unit (12) for heating and cooling the reaction chamber;
an optical detection unit (20) for measuring a nucleic acid amplification reaction, comprising, a light source (21), one or more filters (27, 28), a mirror (23, 24), a focusing lens (22) for transmitting an excitation light (31) irradiated from the light source into the specific reaction chamber (11), a light-receiving lens and a photodetector (26), characterized in that the mirrors (23, 24) and the focusing lens (22) are provided so as to be relatively rotatable with each other; and
a control unit (50) connected to the optical detection unit (20) and the nucleic acid amplification reaction unit (10) through bidirectional communication, comprising input and output unit (52) and a central processing unit (51) to analyze and store the detection result of the nucleic acid amplification reaction product transmitted from the optical detection unit (20).

2. The device according to claim 1, wherein the temperature control unit (12) is composed of a thermoelectric element and a metal block to be heated and cooled, and the reaction chamber (11) is accommodated in the metal block.

3. The device according to claim 1, wherein the temperature control unit (12) is composed of a gas supply device for heating and cooling the reaction chamber (11).

4. The device according to claim 1, wherein the light source is an LED light source.

5. The device according to claim 1, wherein the nucleic acid amplification reaction is PCR reaction or isothermal amplification reaction.

6. The device according to claim 1, wherein the nucleic acid amplification reaction of the sample in each reaction chamber is started at different time points.

7. The device according to claim 2, wherein the light source is an LED light source.

8. The device according to claim 2, wherein the nucleic acid amplification reaction is PCR reaction or isothermal amplification reaction.

9. The device according to claim 2, wherein the nucleic acid amplification reaction of the sample in each reaction chamber is started at different.

10. The device according to claim 3, wherein the light source is an LED light source.

11. The device according to claim 3, wherein the nucleic acid amplification reaction is PCR reaction or isothermal amplification reaction.

12. The device according to claim 3, wherein the nucleic acid amplification reaction of the sample in each reaction chamber is started at different.
